SEQUENCE LISTING (8 sequences)

SEQ ID NO: 1 - DNA, 83 bp, Homo sapiens
taggtaggtgctgctgggtgcccctggagccaaccccagccccatctggaggctccacacccattcccccacctcacattgct

SEQ ID NO: 2 - DNA, 94 bp, Homo sapiens
tcaggtgtgcgagggcgtgggcttcctcctggtgaggtgggggcagggggaggggcagggaagcagaggccctgacccactgtctgtgccttct

SEQ ID NO: 3 - DNA, 126 bp, Homo sapiens
tggtatgggatttacctgataacatttaagaattaaggtgccttagctttccttctctgtctaaacacaaaagtgctaaacataaatttaaactgcttgcggggggatgtgtgatttaaattttat

SEQ ID NO: 4 - DNA, 13 bp, Homo sapiens
ccagtacgtcctc

SEQ ID NO: 5 - DNA, 91 bp, Homo sapiens
gctgtacccaatgggatggcaaggttgtgagccttggataaacccctctgcccttgcttccacacccctctcccagcctgtgccactcacg

SEQ ID NO: 6 - DNA, 10 bp, Homo sapiens
ccttattgac

SEQ ID NO: 7 - DNA, 48 bp, Homo sapiens
ccattgcactgggctgctgtctctgtgttctggcaccagtagcttggg

SEQ ID NO: 8 - DNA, 93 bp, Homo sapiens
actacataagagggcagcgggctcaacgctggcagggccctcccagacccaagaggggcaccatcctctcccgcaccacacccagcgctcaca

(19) United States
(12) Patent Application Publication
(10) Pub. No.: US 2022/0333206 A1
(43) Pub. Date: Oct. 20, 2022

(54) BIOMARKER FOR DIAGNOSING PANCREATIC CANCER, AND USE THEREOF

(71) Applicants: SEOUL NATIONAL UNIVERSITY HOSPITAL, Seoul (KR); SAMSUNG LIFE PUBLIC WELFARE FOUNDATION, Seoul (KR)

(72) Inventors: Youngil KOH, Seoul (KR); Sung-Soo YOON, Seoul (KR); Seulki SONG, Seoul (KR); Joo Kyung PARK, Seoul (KR); Jong Kyun LEE, Seoul (KR); Kyu Taek LEE, Gyeonggi-do (KR); Kwang Hyuck LEE, Seoul (KR); Hyemin KIM, Seoul (KR); Eun Mi LEE, Gyeonggi-do (KR)

(21) Appl. No.: 17/631,597

(22) PCT Filed: Jul. 29, 2020
(86) PCT No.: PCT/KR2020/010014

§ 371 Date: Jan. 31, 2022

(30) Foreign Application Priority Data
Jul. 29, 2019 (KR) 10-2019-0091737
Jul. 29, 2020 (KR) 10-2020-0094635

(51) Int. Cl. C12Q 1/6886

(52) U.S. Cl. CPC C12Q 1/6886; C12Q 2600/158; C12Q 2600/156

(57) ABSTRACT

A method for diagnosing a risk of pancreatic cancer according to an embodiment of the present disclosure includes detecting mutation or functional decrease of one or more gene selected from a group consisting of ARSA (arylsulfatase A), CTSA (cathepsin A), GAA (acid alpha-glucosidase), GALC (galactosylceramidase), HEXB (hexosaminidase subunit beta), IDUA (iduronidase), MAN2B1 (mannosidase alpha class 2B member 1), NPC1 (NPC intracellular cholesterol transporter 1) and PSAP (prosaposin) from a biological sample of a subject, and determining that there is a higher risk of the pancreatic cancer when the mutation or functional decrease of the one or more gene is detected than when neither mutation decrease nor functional decrease is detected.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2020/010014, filed Jul. 29, 2020, which claims priority to the benefit of Korean Patent Application No. 10-2019-0091737, filed Jul. 29, 2019, and Korean Patent Application No. 10-2020-0094635, filed Jul. 29, 2020, the entire contents of which are incorporated herein by reference.

### BACKGROUND

### 1. Technical Field

The present disclosure relates to a novel biomarker for diagnosing pancreatic cancer.

### 2. Background Art

Lysosomal storage diseases (LSDs) are a group of over 50 inherited metabolic disorders that result from defects in the function of endosomal/lysosomal proteins. In LSDs, the defects of genes encoding lysosomal hydrolases or transporters and enzyme activators induce accumulation of macromolecules in the late endocytic system. The disruption of lysosomal homeostasis leads to increased endoplasmic reticulum and oxidative stress, which not only is a mediator of apoptosis in LSDs but also induces oncogenic cellular phenotype and promotes the development of malignancy.

Typical LSD patients have severely impaired organ functions and short life expectancy. However, a considerable number of undiagnosed LSD patients have mildly impaired lysosomal function and survive into adulthood. These patients are often diagnosed after they develop secondary diseases such as Parkinsonism, etc. which are attributable to insidious LSDs.

Clinical observations have shown that patients with Fabry disease or Gaucher disease are at increased risk of cancer, indicating that dysregulated lysosomal metabolism may contribute to carcinogenesis. However, the precise relationship between lysosomal dysfunction and cancer remains unclear. In addition, nonspecific phenotypes result difficulty in recognizing cancer in LSD patients with mild symptoms. Furthermore, the extensive allelic heterogeneity and the complex genotype-phenotype relationships make the cancer diagnosis more challenging. Recent studies suggest that single allelic loss related with LSDs is functionally significant, even though the impact may not be sufficient to develop cancer.

### SUMMARY

The inventors of the present disclosure have analyzed the comprehensive association between germline mutations in lysosomal storage disease-related genes and cancer using data from global sequencing projects. They have identified that carriers of potentially pathogenic variants (PPVs) in 42 lysosomal storage disease-related genes are at increased risk of cancer, the risk of cancer is higher in individuals with a greater number of PPVs, and cancer develops earlier in the PPV carriers. In addition, through whole exome sequencing of Asian pancreatic cancer patients, they have confirmed that 9 among the 42 lysosomal storage disease genes, i.e., ARSA, CTSA, GAA, GALC, HEXB, IDUA, MAN2B1, NPC1 and PSAP, particularly increase the risk of pancreatic cancer.

In addition, they have found that transcriptional misregulation of cancer-promoting signaling pathways might underlie the oncogenic contribution of PPVs and completed the present disclosure by revealing potential mechanisms that might be involved in oncogenesis through analysis of tumor genomic and transcriptomic data from pancreatic adenocarcinoma.

The present disclosure is directed to providing a method for providing information for diagnosing cancer using a lysosomal storage disease-related gene as a biomarker.

However, the technical problem to be solved with the present disclosure is not limited to that described above and other unmentioned problems will be clearly understood by those having ordinary skill in the art.

The present disclosure provides a biomarker composition for diagnosing or predicting pancreatic cancer, which includes mutation of one or more gene selected from a group consisting of ARSA (arylsulfatase A), CTSA (cathepsin A), GAA (acid alpha-glucosidase), GALC (galactosylceramidase), HEXB (hexosaminidase subunit beta), IDUA (iduronidase), MAN2B1 (mannosidase alpha class 2B member 1), NPC1 (NPC intracellular cholesterol transporter 1) and PSAP (prosaposin).

In addition, the present disclosure provides a composition for diagnosing or predicting pancreatic cancer, which contains an agent capable of detecting mutation of one or more gene selected from a group consisting of ARSA, CTSA, GAA, GALC, HEXB, IDUA, MAN2B1, NPC1 and PSAP.

In an exemplary embodiment of the present disclosure, the mutation is non-silent mutation, and the mutation may be nonsense mutation, missense mutation or frameshift mutation whereby the function of a protein encoded by the gene declines as a result of substitution, insertion and/or deletion of the base pairs of the gene.

In another exemplary embodiment of the present disclosure, the composition may be for diagnosing or predicting pancreatic cancer in Asians, particularly for diagnosing or predicting pancreatic cancer in Koreans, although not being limited thereto.

In another exemplary embodiment of the present disclosure, the agent may be one or more selected from a group consisting of an oligonucleotide, a primer, a probe and a compound binding specifically to the gene.

In addition, the present disclosure provides a kit for diagnosing or predicting pancreatic cancer, which includes the composition.

In addition, the present disclosure provides a method for providing information necessary for diagnosing the risk of pancreatic cancer and a method for diagnosing the risk of pancreatic cancer, which include a step of detecting mutation of one or more gene selected from a group consisting of ARSA (arylsulfatase A), CTSA (cathepsin A), GAA (acid alpha-glucosidase), GALC (galactosylceramidase), HEXB (hexosaminidase subunit beta), IDUA (iduronidase), MAN2B1 (mannosidase alpha class 2B member 1), NPC1 (NPC intracellular cholesterol transporter 1) and PSAP (prosaposin) from a biological sample of a subject.

In an exemplary embodiment of the present disclosure, the method for diagnosing and the method for providing information may further include, after the step of detecting mutation of the gene, a step of determining that there is a high risk of pancreatic cancer when the mutation of the gene is detected.

In another exemplary embodiment of the present disclosure, the method for diagnosing and the method for providing information may further include a step of determining that the risk of pancreatic cancer is about 5 times higher when there is mutation in the GALC gene as compared to a normal group with no mutation.

In another exemplary embodiment of the present disclosure, the method for diagnosing and the method for providing information may further include a step of determining that the risk of pancreatic cancer is 2 times higher when mutation is detected in two or more genes selected from a group consisting of ARSA, CTSA, GAA, GALC, HEXB, IDUA, MAN2B1, NPC1 and PSAP.

In another exemplary embodiment of the present disclosure, the biological sample may be a cell sampled from the blood or cancerous tissue of the subject, although not being limited thereto.

In another exemplary embodiment of the present disclosure, the detection of mutation of the gene may be performed by one or more method selected from a group consisting of measurement of the activity of an enzyme encoded by the gene, measurement of the expression level of the gene and gene sequencing, and the measurement of the expression level of the gene may be performed by gene amplification or microarray methods.

The inventors of the present disclosure have elucidated the association between potentially pathogenic germline mutations in lysosomal storage disease-related genes and pancreatic cancer, thereby enabling early diagnosis and management of pancreatic cancer. In addition, the present disclosure provides a platform for designing customized strategy for prevention and treatment of pancreatic cancer through detection of a pancreatic cancer-related biomarker and thus provides a target for prevention and treatment of pancreatic cancer.

### BRIEF DESCRIPTION OF THE DRAWINGS

FIG. 1 shows the PPV selection criteria and population composition of Pan-Cancer and 1,000 Genomes cohorts. The populations of the Pan-Cancer cohort (a of FIG. 1) and the 1,000 Genomes cohort (b of FIG. 1), the population of the Pan-Cancer cohort constituting each type of cancer (c of FIG. 1), and a Venn diagram of PPVs identified in the Pan-Cancer and 1,000 Genomes cohorts grouped into three tiers (d of FIG. 1) are shown.

FIG. 2 shows PPVs occurring with significantly high frequencies in cancer patients. a of FIG. 2 shows odds ratios for the prevalence of single, double and triple PPV carriers with or without population adjustment, and b of FIG. 2 shows odds ratios for the prevalence of RSVs analyzed in the same manner as for the PPVs. Error bars indicate 95% confidence intervals.

FIG. 3 shows the numbers of PPV carriers (a of FIG. 3) and RSV carriers (b of FIG. 3) for 41 LSD genes found in the Pan-Cancer cohort and the 1,000 Genomes cohort.

FIG. 4A shows the SKAT-O association between 30 major histological types of cancer (>15 patients per type) and PPVs in each LSD gene, and FIG. 4B shows the Q-Q plot of P values derived from SKAT-O analysis.

FIG. 5 shows odds ratios and 95% confidence intervals for PPV carriers in eight cancer patient cohorts versus an ExAC control cohort.

FIGS. 6A to 6F show age at diagnosis of cancer. FIG. 6A shows the age at diagnosis of cancer in 28 major clinical cancer cohorts, FIG. 6B shows the age at diagnosis of cancer in PPV carriers and non-carriers in the Pan-Cancer cohort and six clinical cancer subgroups that showed significant SKAT-O association with PPVs, FIG. 6C shows the age at diagnosis of cancer according to the carrier status of 11 PPV groups significantly associated with the Pan-Cancer cohort or more than two histological cancer subgroups in the SKAT-O analysis, FIG. 6D shows the linear correlation between the PPV load and the age at diagnosis of cancer in the six clinical cancer subgroups shown in FIG. 6B, FIG. 6E shows the linear correlation between the PPV load and the age at diagnosis of cancer in the Pan-Cancer cohort for each of the 11 PPV groups shown in FIG. 6B, and FIG. 6F shows all-gene pairs in which the age at diagnosis of cancer differs significantly according to the PPV carrier status.

FIG. 7 shows nonsynonymous somatic mutations in the 50 most frequently mutated genes in pancreatic adenocarcinoma tissues obtained from PPV carriers (n=55, left panel) and PPV non-carriers (n=177, right panel) who are patients with pancreatic adenocarcinoma.

a to c of FIG. 8 show a DEG analysis result showing 287 gene upregulations and 221 gene downregulations in PPV-associated pancreatic adenocarcinoma, d of FIG. 8 is a heatmap showing the relative expression of genes significantly up- or downregulated at the 0.1 FDR threshold in tumors from PPV carriers versus PPV non-carriers, and e of FIG. 8 shows the KEGG ways that are significantly altered in tumors from PPV carriers compared with those from PPV non-carriers.

FIG. 9 shows the statistical significance of the difference in the number of PPV carriers in a cohort of Asian pancreatic cancer patient and a control cohort of healthy Korean people. The statistical significance for the GALC gene in lysosomal storage disease and the significance for total lysosomal storage disease genes are shown.

FIGS. 10A and 10B show the process whereby cancer occurs in carriers of lysosomal storage disease genes. FIG. 10A shows that the possibility of occurrence of two hits in the BRCA gene owing to somatic mutation in cancer cells of lysosomal storage disease gene carriers is significantly higher as compared to other genes. FIG. 10B shows that loss of heterozygosity (LOH) occurs due to copy number loss in mutation sites of organoids and germline mutations (carrier status) in actual pancreatic cancer patients (FIG. 10B).

FIGS. 11A and 11B show that the expression level of lysosomal storage disease genes is decreased when PPV and LOH occur at the same time in the organoids of pancreatic cancer patients.

### DETAILED DESCRIPTION

Hereinafter, the present disclosure is described in more detail.

In an aspect, the present disclosure provides a biomarker for diagnosing or predicting pancreatic cancer, which includes mutation of a lysosomal storage disease-related gene, specifically one or more gene selected from a group consisting of ARSA (arylsulfatase A), CTSA (cathepsin A), GAA (acid alpha-glucosidase), GALC (galactosylceramidase), HEXB (hexosaminidase subunit beta), IDUA (iduronidase), MAN2B1 (mannosidase alpha class 2B member 1), NPC1 (NPC intracellular cholesterol transporter 1) and PSAP (prosaposin).

The gene may have a decreased activity of a protein encoded by the gene as compared to the wild type due to amino acid substitution, deletion and/or insertion, and may exhibit the carrier (potentially pathogenic variant) phenotype owing to the mutation.

In another aspect, the present disclosure provides a composition for diagnosing or predicting pancreatic cancer, which contains an agent capable of detecting the mutation of one or more gene selected from a group consisting of ARSA, CTSA, GAA, GALC, HEXB, IDUA, MAN2B1, NPC1 and PSAP.

In a specific exemplary embodiment of the present disclosure, the agent may be an antisense oligonucleotide binding specifically to the gene, and the antisense oligonucleotide may be a primer pair or a probe, although not being limited thereto.

In another aspect, the present disclosure provides a method for providing information necessary for diagnosing the risk of pancreatic cancer, which includes: a step of detecting mutation of one or more gene selected from a group consisting of ARSA, CTSA, GAA, GALC, HEXB, IDUA, MAN2B1, NPC1 and PSAP in a subject; and a step of determining the there is a high risk of pancreatic cancer when the mutation of the gene is detected.

5-10% of pancreatic cancer patients are diagnosed at ages before 50 years. Family history is a strong risk factor in pancreatic cancer patients, which suggests the presence of hereditary risky mutation. Mutation of genes involved in DNA double strand break repair (e.g., BRCA1/2 or PALB2) has been confirmed in many pancreatic cancer patients. However, the genetic cause of early onset of pancreatic cancer has not been elucidated in most patients. In the histospecific analysis of the present disclosure, pancreatic adenocarcinoma patients showed strong association with PPV of some LSD genes. A tendency of early onset was shown in the patients in which PPV was found. The difference in somatic mutation and gene expression pattern was confirmed in the histological types. Up- or downregulations of many PPV-associated genes were confirmed through DEG analysis, and the biological pathways that may be involved in the onset of pancreatic cancer in the patients were analyzed by GAGE analysis. Many of the altered pathways identified in the GAGE analysis were previously implicated in pancreatic cancer development in transcriptome and exome sequencing studies. The somatic mutation burden and signatures, in contrast, were comparable between the carriers and non-carriers of PPV. Overall, the present disclosure suggests that transcriptional misregulation is a key mediator of pancreatic carcinogenesis triggered by PPVs.

The “two-hit hypothesis” is the hypothesis that cancer occurs as both alleles lose their function due to inactivation. It is important in that carcinogenesis in carriers of specific heterozygotes can be explained. In order to confirm whether the biomarker of the present disclosure conforms to the hypothesis, the inventors of the present disclosure have compared LOH with known cancer predisposition genes using Alfred's method and have obtained a statistically significant result.

From a therapeutic aspect, LSD genes are attractive targets because of the mechanically intuitive nature of enzyme replacement and substrate reduction therapies. The enzyme replacement therapy has already been approved for at least seven types of LSD. Other promising approaches include pharmacological chaperones, gene therapy and compounds that read through the early stop codon introduced by nonsense mutations. Although it is unclear whether preemptive treatment can prevent or delay long-term complications of LSD, the present disclosure makes it promising to harness the LSD therapy for preventing cancer in carriers of inactivating germline mutations in LSD genes. That is to say, the present disclosure provides a comprehensive landscape of the association potentially pathogenic germline mutations in LSD genes and cancer. Investigating the relationship between treatable metabolic diseases and cancer is crucial since it can build the basis for precise cancer prevention. Diverse therapeutic options to restore lysosomal function are being developed currently. Further clinical trials of these agents guided by individuals' mutation profiles may pave a new path toward personalized cancer prevention and treatment.

The present disclosure can be changed variously and may have various exemplary embodiments. Hereinafter, specific exemplary embodiments will be described in detail referring to drawings. However, it should be understood that the present disclosure is not limited by the specific exemplary embodiments but include all modifications, equivalents or substitutes encompassed within the technical idea and scope of the present disclosure. When describing the present disclosure, detailed description of known technology will be omitted if it unnecessarily obscures the subject matter of the present disclosure.

### [Methods]

### 1. Data Sources

Germline and somatic (tumor) variant datasets for single nucleotide variants (SNVs) and indels (insertions and deletions) of the Pan-Cancer cohort were downloaded as VCF and MAF format files, respectively, from the SFTP server of the PCAWG project. The germline variant datasets encompassed 2,834 PCAWG donors and were produced using the DKFZ/EMBL pipeline. The tumor somatic MAF file contained data of 2,583 whitelist samples (only one representative tumor from each multi-tumor donor) and was generated by the PCAWG consensus strategy consolidating outputs from the Sanger, Broad, DKFZ/EMBL and MuSE pipelines for SNVs and from the SMuFin, DKFZ, Sanger and Snowman pipelines for indels.

Pass-only variants were used for the analysis. Tumor RNA-Seq data were downloaded as both raw and normalized read count matrices of protein-coding genes via Synapse. Read alignment was carried out using TopHat2, counted using the HTSeq-count script from the HTSeq framework version 0.61p1 against the reference General Transfer Format of GENCODE release 19, and normalized using the FPKM-UQ normalization technique. Clinical and histological annotation sheets were downloaded from the PCAWG wiki page in version 9 (generated on Nov. 22, 2016 and Aug. 21, 2017, respectively).

As a primary control cohort, individual-level data of SNVs and insertion-deletion genotypes for 2,504 individuals were downloaded from the 1,000 Genomes project phase 3 as VCF files. In addition, population-level AF data for SNVs and indels for 53,105 unrelated individuals from the ExAC release 1.0 (ExAC cohort), excluding TCGA subset, were downloaded for use as an independent validation control.

### 2. Quality Assessment and Control

Quality assessment of all PCAWG sequence data was carried out according to three-level criteria (library, sample and donor levels) to determine whether to include each donor and RNA-Seq aliquot or not. This multi-level quality control process is necessary since individual donors can have multiple samples and individual samples can have multiple libraries. As a rule, a sample was blacklisted if all of its libraries were of low quality, and whitelisted if all of its libraries were of high quality. Similarly, a donor was blacklisted if all associated samples were blacklisted, and whitelisted if all associated samples were whitelisted. Samples and donors that were neither blacklisted nor whitelisted were included in graylisted. Only whitelisted individuals and samples (2,583 tumor-normal pair genomes and 1,094 RNA-Seq samples) were included in the study. Quality control criteria for each level of assessment are detailed in the PCAWG marker paper.

### 3. Consolidation of Pan-Cancer Cohort

The original PCAWG project covered 2,834 individuals encompassing 40 major cancer types as part of the ICGC, which included 76 projects and 21 primary organ sites. Among those, 2,583 whitelisted patients who satisfied the multi-level quality control criteria were prioritized. 16 patients diagnosed with benign bone neoplasm such as chondroblastoma, chondromyxoid fibroma, osteofibrous dysplasia and osteoblastoma were excluded, leaving 2,567 patients in the Pan-Cancer cohort.

Nine patients who had multiple tumor specimens were associated with more than one histological diagnosis: eight with myeloproliferative neoplasm and acute myeloid leukemia, and one with hepatocellular carcinoma and cholangiocarcinoma. For consistency in the histology-specific analysis, the first eight patients were classified as acute myeloid leukemia and the ninth patient as cholangiocarcinoma. To analyze the age at diagnosis of cancer, multiple histological cohorts that shared similar clinicopathologic characteristics were combined into a single clinical cohort (e.g., breast-invasive ductal, lobular and microcapillary carcinomas were classified as breast cancer, and myeloproliferative neoplasm and myelodysplastic syndrome as chronic myeloid disorder). Among the 2,567 patients, only 1,075 had whitelisted tumor RNA-Seq data. Since 19 patients contributed more than one tumor specimen, RNA-Seq data were available for 1,094 tumors.

### 4. Gene Selection and Variant Interpretation

Of the genes involved in lysosomal functions that include substrate hydrolysis, post-translational modification of hydrolases, intracellular trafficking, enzymatic activation, etc., 42 genes that were previously implicated in the development of LSD were selected via literature review (Parenti, G., Andria, G. & Ballabio, A. Lysosomal storage diseases: from pathophysiology to therapy. Annu. Rev. Med. 66, 471-486 (2015); Wang, R. Y., Bodamer, O. A., Watson, M. S. & Wilcox, W. R. Lysosomal storage diseases: Diagnostic confirmation and management of presymptomatic individuals. Genet. Med. 13, 457-484 (2011); Scriver, C. R. The metabolic and molecular bases of inherited disease, (McGraw-Hill, New York, 2001); Boustany, R.-M. N. Lysosomal storage diseases—the horizon expands. Nature Reviews Neurology 9, 583-598 (2013); and Futerman, A. H. & van Meer, G. The cell biology of lysosomal storage disorders. Nat. Rev. Mol. Cell Biol. 5, 554-565 (2004)).

The genomic loci of the selected genes based on the GRCh37/hg19 human reference genome assembly were screened for all germline SNVs and indels in each VCF file. Variants were identified based on the GENCODE release 19 gene model. Functional annotation was carried out using both ANNOVAR and Variant Effect Predictor version 85. The outputs were cross-checked and manually curated to achieve the most appropriate characterization of each identified variant. The analysis focused on variants within protein-coding regions and splice donor and acceptor sites within two base pairs to the intron side from the exon-intron junctions (GT-AG conserved sequence) and 5′ and 3′ untranslated regions (UTRs).

Variants were classified into ten non-overlapping categories according to the predicted consequence type on transcripts or proteins (missense, start-loss, stop-gain, stop-loss, synonymous, frameshift indel, non-frameshift indel, splicing, and 5′ and 3′ UTR variants). When a variant was associated with more than one consequence type depending on transcript isoforms, it was classified into the most functionally disruptive category (e.g., protein-truncating rather than missense, and missense rather than UTR or synonymous). For example, rs373496399 (NC_000017.10: g.78184457G>A) could be either a missense or 3′ UTR variant depending on the transcript isoform and was classified as missense. By this way, each variant belonged to a unique functional class that was used for subsequent analysis. In silico prediction of the mutational effect on protein function was carried out by using 19 distinct computational algorithms with the use of dbNSFP version 3.3.

### 5. PPV Selection

The prevalence of individual LSDs ranges from one per tens of thousands to one in millions of live births, and considerable allelic heterogeneity exists. Therefore, a single variant with a population AF≥0.5% is extremely unlikely to be causative, even when considering the possibility of underdiagnosis. A recent analysis of the prevalence of known Mendelian disease variants using >60,000 exomes sequenced suggested that a substantial proportion of variants with AF>1% were, in fact, benign or functionally neutral, highlighting the importance of filtering PPVs based on their frequency in a sufficiently large reference population. On this theoretical basis and experimental data showing that deleterious variants were rare, mostly with an AF of <0.5%, variants with an average AF between the Pan-Cancer and 1000 Genomes cohorts of 0.5% were excluded during the PPV selection process.

Curated databases were examined using ClinVar, HGMD, and LSMDs and medical literatures described in Table 1 were reviewed extensively to identify LSD-causing mutations.

TABLE 1

| HGNC | |
|---|---|
| Symbol | Database |
| GBA | Leiden Open Variation Database |
| HEXA | HEXdb |
| GAA | Leiden Open Variation Database |
| IDUA | Leiden Open Variation Database |
| HGSNAT | Leiden Open Variation Database |
| GLA | Leiden Open Variation Database |
| IDS | Leiden Open Variation Database |
| PPT1 | NCL Mutation and Patient Database |
| TPP1 | NCL Mutation and Patient Database |
| CLN3 | NCL Mutation and Patient Database |
| | Retina International's Scientific Newsletter |

Initially, variants were classified into five non-overlapping categories, as proposed by the American College of Medical Genetics and Genomics (ACMG) and Association for Molecular Pathology (AMP) based on the curated clinical significance information in ClinVar. In case of variants that belonged to more than one pathogenicity category, priority was assigned to the category associated with stronger evidence, hence ‘benign’ rather than ‘likely benign,’ and ‘pathogenic’ rather than ‘likely pathogenic.’ When interpretations indicating both pathogenic (‘pathogenic’ or ‘likely pathogenic’) and benign (‘benign’ or ‘likely benign’) directions of effect coexisted for a single variant, or no pathogenicity interpretation was provided in standard terminology, data in HGMD and LSMDs along with supporting evidence obtained from direct literature survey were reviewed to determine the most relevant functional category of the variant according to the ACMG and AMP guideline.

The role of microRNA in carcinogenesis has been spotlighted in recent years. In the present disclosure, it was identified that many SNVs in 3′ UTR microRNA-binding sites are involved in the increased or decreased cancer risk via altered expression of gene products. In addition, it was identified that 5′ UTRs also contain binding motifs for microRNAs, and their sequence variation affects messenger RNA (mRNA) stability. Since UTR variants can create or destroy a microRNA-binding motif that regulates gene expression and mRNA degradation, the biological consequence of UTR variants can be reflected in the change in transcript abundance in relevant tissues.

Therefore, RNA-Seq read count data were analyzed to identify UTR variants associated with significantly decreased expression of the corresponding genes. Among the 3,192 unique UTR variants with mean AF<0.5% between the Pan-Cancer and 1000 Genomes cohorts, 795 and 2,397 were present in 5′ and 3′ UTRs, respectively. Tissue mRNA abundance was compared after variance-stabilizing transformation of read counts between UTR variant carriers and non-carriers for each gene, using linear regression. Because the expression level of each LSD gene varied considerably across cancer types, the regression model was adjusted for cancer histology. As a result, only one 3′ UTR variant in IDS rs145834006 reached statistical significance at the 0.1 FDR threshold.

After inspection of all information obtained from the above processes, PPVs that were highly likely to cause LSD were selected by using three positive selection criteria. Tier 1 included all frameshift indels, start-loss variants, stop-gain variants, splicing variants, and a UTR variant associated with significant downregulation of the corresponding gene (rs145834006). Thus, most of these variants were loss-of-function in principle. Tier 2 included variants classified as ‘pathogenic’ or ‘likely pathogenic’ based on the information obtained from ClinVar and relevant medical literature, disease-causing mutations in HGMD.

Of the variants without curated pathogenicity information in both ClinVar and HGMD (i.e., with unknown clinical significance), those predicted to be functionally deleterious by all of the 19 separate in silico prediction tools were classified into tier 3. The score threshold of each tool for classifying a variant as deleterious or benign was set at the provided default when available, or the median of all evaluated variants otherwise. Because some variants (especially those in the noncoding regions and indels) were not successfully annotated by all of the 19 tools, only available scores were used in such cases.

### 6. PPV-Cancer Association Analysis Using Pan-Cancer and 1,000 Genomes Cohorts

Because the cohorts were underpowered to detect variant-specific associations for such rare variants as PPVs, tier- and gene-based aggregate association analysis was performed using the SKAT-O method with an optimal p parameter chosen from a grid of eight points (0, 0.12, 0.22, 0.32, 0.42, 0.52, 0.5 and 1), which could be interpreted as a pairwise correlation among the genetic effect coefficients. The SKAT-O method is robust against the co-existence of pathogenic and benign variants and is thus suitable when no uniform assumption can be made for the genetic effects of variants.

To examine if the difference in variant calling pipelines used in the PCAWG project and the 1000 Genomes project (batch effects) affected the results, the PPV-to-synonymous variant prevalence ratios were compared between cancer cohorts and the 1000 Genomes cohort using weighted logistic regression. For an exploratory purpose, the variant-specific association of PPVs with each type of cancer using logistic regression was also assessed assuming a multiplicative risk model. All association analyses were adjusted for population structure using the method described below.

### 7. Population Structure Adjustment

For adjustment of population structure, principal component analysis was carried out using the individual-level genotype data of tag single nucleotide polymorphisms (tag-SNPs) of the Pan-Cancer and 1000 Genomes cohorts. First, a list of 1,555,886 candidate tag-SNPs was downloaded from the phase 3 HapMap ftp server. The genomic coordinates of these SNPs were converted into the GRCh37/hg19 framework using the Batch Coordinate Conversion (liftOver) tool. VCF files from both the Pan-Cancer and 1000 Genomes cohorts were merged using the Genome Analysis Toolkit to calculate broad AFs.

VCFtools version 1.13 was used to extract candidate tag-SNPs with AF≥5% and ≤50% from the merged VCF, leaving 16,304 SNPs in the aggregate genotype matrix. Among those, the population-stratifying tag-SNPs were prioritized using the PLINK pruning method. During this process, a recursive sliding-window procedure was used to exclude SNPs with a variance inflation factor>5 within a sliding window of 50 SNPs, shifting the window forward by 5 SNPs at each step. As a result, the linkage disequilibrium panels containing multiple correlated SNPs were reduced to 10,494 representative tag-SNPs, which were used in the subsequent principal component analysis.

A total of 5,071 principal components (PCs) were obtained by performing principal component analysis against the combined genotype data for the 10,494 tag-SNPs of the Pan-Cancer and 1000 Genomes cohorts. The correlations of each PC with the binary phenotype (cancer versus normal) and PPV load were calculated. Predictably, PC1 and PC2 collectively accounted for more than 11% of the total variance and only these two were significantly correlated with both the binary phenotype and PPV load at the 0.1 FDR threshold. The remaining 5,069 PCs accounted for less than 1% of the variance and were correlated with either the phenotype or the PPV load or neither, suggesting that only the two top-ranked PCs were potential confounders of the association between PPVs and cancer.

Therefore, PC1 and PC2 were included as covariates in the subsequent association analyses. To examine the possibility of systematic inflation of test statistics, a group-based inflation factor (λ) was calculated from the histology-specific SKAT-O results using the method described above.

### 8. RNA-Seq Data Analysis

The genes with zero read counts across all tumors were filtered out from the read count matrices to improve the computational speed. Since the data were generated on the framework of Ensembl gene classification, the Ensembl gene ID was converted to Entrez gene ID using Pathview. When multiple Ensembl IDs matched to a single Entrez ID, those with the largest variance across all samples were selected while the others were removed from the count matrix.

The differential gene expression patterns between tumors from PPV carriers and non-carriers were investigated using DESeq2, after applying the shrinkage estimation of log fold changes and dispersions to improve the stability of the estimates. Before estimating FDRs for DEG results, independent filtering of low-count genes was performed using Genefilter to improve statistical power.

Before the GAGE analysis, variance-stabilizing transformation of raw read counts was performed to achieve homoscedasticity of the count matrix and decrease the influence of genes with an excessively large variation in expression level across samples. The GAGE analysis was based on group-on-group comparisons, which could be controlled by the ‘compare’ argument supported by the ‘gage’ function of the Bioconductor package ‘gage.’ The upregulation and downregulation of gene components constituting the Kyoto Encyclopedia of Genes and Genomes (KEGG) pathways in tumors from PPV carriers compared to those from non-carriers were tested simultaneously.

### 9. Validation Analysis Using ExAC Cohort as Independent Control

Because the ExAC cohort dataset covered only exomic regions consisting of the GENCODE release 19 coding regions and their flanking 50 base pairs, analysis was restricted to coding regions covered in more than half of the ExAC samples (median coverage depth 1) in the validation analysis. Coverage depth for the ExAC sequence data was downloaded from the ftp site. Then, PPVs were selected from the aggregate variant call set of the Pan-Cancer and ExAC cohorts using the same criteria used in the primary analysis of the Pan-Cancer and 1000 Genomes cohorts.

As a result, 1,267 PPVs were identified: 942 in tier 1 and 475 in tier 2 with 150 overlaps between the two tiers. No tier 3 PPV was identified because the pathogenicity score thresholds used for classifying each variant as deleterious or neutral were set at stricter values than in the primary analysis for some of the 19 in silico prediction tools. The changes in thresholds were owing to the algorithmic decision to set the thresholds at medians of the scores derived from all evaluated variants identified in the Pan-Cancer and ExAC cohorts, which differed from the median values of variants identified in the Pan-Cancer and 1000 Genomes cohorts.

Although the TCGA subset was excluded from the ExAC cohort to avoid contamination of the control with cancer patients, a large portion of the ExAC cohort was comprised of individuals with diseases that might be associated with LSD-causing mutations (e.g., schizophrenia and bipolar disorder). The mean PPV frequency varied considerably across populations in the ExAC cohort, and correlations between the PPV frequencies of different populations were relatively low for the East Asian and African populations.

### 10. Statistical Analysis of ICGC-PCAWG Data

A two-step approach was employed to examine the association between PPVs and cancer. In the first step, the Pan-Cancer and 1000 Genomes cohorts were analyzed with the SKAT-O method for the aggregate rare-variant association and Fisher's exact tests and logistic regressions for direct comparison of mutation prevalence. The Cochran-Armitage trend test was used to evaluate the association between cancer risk and PPV load. Population structure was adjusted through principal component analysis on 10,494 tag-SNPs.

In the second step, the ExAC cohort was used an independent control and Fisher's exact test was performed to validate the preceding results. The age at diagnosis of cancer was compared using Wilcoxon rank-sum test and linear regression. DEG and gene set analyses were performed using the DESeq2 Bioconductor package and the GAGE method based on the framework of KEGG pathways, respectively.

Correction for multiple testing was conducted using the FDR estimation procedure (tail area-based FDR (q-value)). All tests were two-tailed unless specified otherwise. FDR<0.1 and P<0.05 (when not adjusted for multiple testing) were considered significant. Statistical analysis was performed using R software, version 3.5.0 with packages of Bioconductor version 3.7.

### 11. Whole Exome Data Analysis: PPV and Two-Hit Analysis

A Korean clinical cohort was established for validation of carcinomas highly with high association between PPVs and cancer based on the large-scale genomic data. For pancreatic cancer, whole exome sequencing data were generated for a total of 214 samples with a mean coverage 50 for detection of exact germline variations. QC (quality control) was performed for all variants to avoid pseudovariation occurring due to biases during NGS (next-generation sequencing). Phred-scaled probability values, which are thought be depth, strand information and bias, were calculated and filtered for all the variants detected for all samples. Through this, wrongly extracted variants or strand biases occurring frequently in exon edge could be removed. Variant filtering was carried out using various variant score indices such as QD (quality depth), FS (allele-specific phred-scaled p-value), MQ (mapping quality), MQRankSum (mapping quality rank sum), ReadPosRankSum (rank sum test of Alt vs. Ref), etc. The filtering was performed by applying different variant score indices depending on the characteristics of the genomic data. For WGS and WES with broad sequencing target regions, VQSR (variant quality recalibration) was applied to score indices corresponding to known variants in 1000G, HapMap, dbSNP, etc. using machine learning. The filtering was performed based on the GATK WES criteria, and a more reasonable cut-off was used according to the genomic data status to minimize errors depending on the cohort characteristics. Only canonical transcripts were extracted from the extracted variants using ANNOVAR and Ensembl's Variant Effect Predictor (VEP), and accurate annotation information such as dbSNP, Clinvar, GnomAD, etc. was added. The Clinvar databases show difference in pathogenicity depending on versions. Clinvar_20190618, which is the newest version, was used. PPVs were screened in the same manner as described above. Because the data generated from Koreans were used for the study of the homogeneous cohort, the PPV screening was performed by adjusting AF to 1% for detection ethnicity-specific rare genetic variants that occurred specifically in the Korean cohort.

### 12. Analysis of Expression Level of Lysosomal Storage Disease Genes in Organoids of Pancreatic Cancer Patients

Analysis was conducted for comparison of the difference in gene expression level in 15 cases of pancreatic cancer depending on the presence of LSD. For this, the generated organoid transcriptomic data were mapped using STAR, RSEM-1.3.0. The carrier gene expression level was compared for all the samples based on the TPM values obtained through normalization depending on the difference in final depth and read depth.

### 13. Statistical Analysis

The association between 42 LSD genes and GALC genes and carcinogenesis was analyzed in the Korean pancreatic cancer patients, and chi-square test was conducted for mutation prevalence using the Korean normal group cohort as an independent control. The transcriptomic analysis of GALC genes depending on the presence of PPV carrier was compared using the expression level of GALC genes with the mean level of 41 LSD genes excluding the same. Statistical significance was investigated by Wilcoxon rank-sum test. The statistical significance was tested using R.

### 14. Data Availability

The data that support the present disclosure are available publicly or with proper authorization. The germline and somatic (tumor) variant call sets and the RNA-Seq read count matrices derived from the PCAWG project are available for general research use under the data access policies of the ICGC and TCGA projects.

In order to gain authorized access to the controlled-tier elements of the data, application to the TCGA Data Access Committee via dbGAP for the TCGA portion and to the ICGC Data Access Compliance Office (DACO) for the remainder is necessary. Clinical and pathological data of individual donors and specimens are in an open tier and are accessible through the ICGC Data Portal. Variant call sets derived from the 1000 Genomes project phase 3 and the ExAC release 1.0 are publicly available at the individual level and the population level, respectively, from the sources described in the Methods.

### [Analysis Results]

### 1. Characteristics of Study Cohorts

Matched tumor-normal pair whole genome and tumor whole transcriptome sequence data and clinical and histological annotation of 2,567 cancer patients (Pan-Cancer cohort) from the International Cancer Genome Consortium (ICGC)/The Cancer Genome Atlas (TCGA) Pan-Cancer Analysis of Whole Genomes (PCAWG) project were used. As controls, publicly available variant call sets from two global sequencing projects of individuals without known cancer histories were used. The first control dataset comprised 2,504 genomes from the 1000 Genomes project phase 3 (1000 Genomes cohort). The second dataset included exomes of 53,105 unrelated individuals from a subset of the Exome Aggregation Consortium release 1.0 that did not include TCGA subset (ExAC cohort).

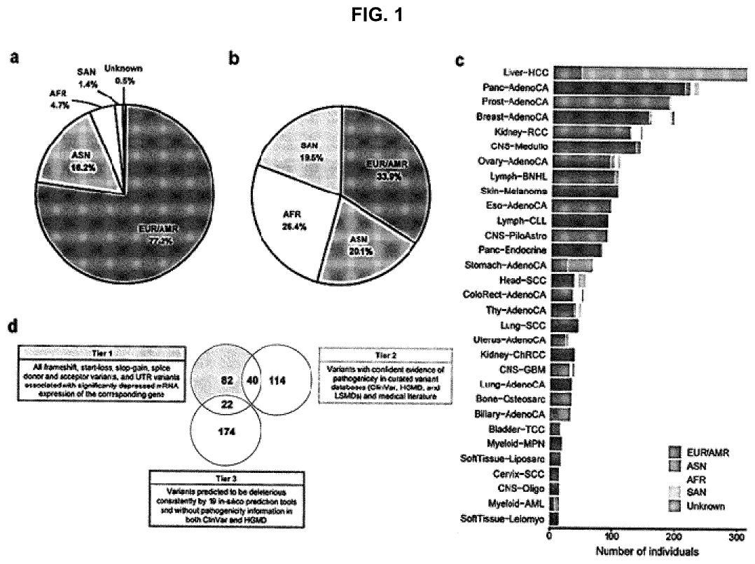

The Pan-Cancer cohort consisted of four populations and 38 histological types of pediatric or adult cancer (a of FIG. 1 and c of FIG. 1). The median age at diagnosis was 60 years (range, 1 to 90 years). A majority of the patients were Europeans or Americans in most cancer types. The 1000 Genomes cohort comprised five populations (b of FIG. 1) and was combined the European and American populations for comparison with the Pan-Cancer cohort. The ExAC cohort included seven populations, among which the Americans and Non-Finnish Europeans together accounted for more than 60% of the entire cohort.

### 2. PPV Prevalence in Pan-Cancer and 1,000 Genomes Cohorts

Through extensive literature review, 42 LSD genes were identified. The LSD genes are listed in Table 2.

TABLE 2

| | Gene | | | |
|---|---|---|---|---|
| Cate- | (HGNC | | | |
| gory | Symbol) | Chromosome | Associated lysosomal storage diseases | Genetic pattern |
| 1 | AGA | 4 | Aspartylglycosaminuria | Autosome formed |
| 2 | ARSA | 22 | Metachromatic leukodystrophy | Autosome formed |
| 3 | ARSB | 5 | Mucopolysaccharidosis VI | Autosome formed |
| | | | (Maroteaux-Lamy syndrome) | |
| 4 | ASAH1 | 8 | Farber lipogranulomatosis | Autosome formed |
| 5 | CLN3 | 16 | Neuronal ceroid lipofuscinosis(NCL) 3 | Autosome formed |
| | | | (juvenile NCL or Batten disease) | |
| 6 | CTNS | 17 | Cystinosis | Autosome formed |
| 7 | CTSA | 20 | Galactosialidosis | Autosome formed |
| 8 | CTSK | 1 | Pycnodysostosis | Autosome formed |
| 9 | FUCA1 | 1 | Fucosidosis | Autosome formed |
| 10 | GAA | 17 | Glycogen storage disease type II | Autosome formed |
| | | | (Pompe disease) | |
| 11 | GALC | 14 | Globoid cell leukodystrophy (Krabbe disease) | Autosome formed |
| 12 | GALNS | 16 | Mucopolysaccharidosis IVA | Autosome formed |
| | | | (Morquio A syndrome) | |
| 13 | GBA | 1 | Gaucher disease | Autosome formed |
| 14 | GLA | X | Fabry disease | X chromosome |
| | | | | formed |
| 15 | GLB1 | 3 | Mucopolysaccharidosis IVB | Autosome formed |
| | | | (GM1 gangliosidosis and Morquio B syndrome) | |
| 16 | GM2A | 5 | GM2-gangliosidosis type AB | Autosome formed |
| 17 | GNPTAB | 12 | Mucolipidosis II (I-cell disease) | Autosome formed |
| | | | Mucolipidosis IIIA (pseudo-Hurler polydystrophy) | |
| 18 | GNPTG | 16 | Mucolipidosis IIIC (mucolipidosis III gamma) | Autosome formed |
| 19 | GNS | 12 | Mucopolysaccharidosis IIID | Autosome formed |
| | | | (Sanfilippo syndrome D) | |
| 20 | GUSB | 7 | Mucopolysaccharidosis VII (Sly syndrome) | Autosome formed |
| 21 | HEXA | 15 | GM2 gangliosidosisi type I (Tay-Sachs disease) | Autosome formed |
| 22 | HEXB | 5 | GM2 gangliosidosis type 2 (Sandhoff disease) | Autosome formed |
| 23 | HGSNAT | 8 | Mucopolysaccharidosis IIIC | Autosome formed |
| | | | (Sanfilipppo syndrome C) | |
| 24 | HYAL11 | 3 | Mucopolysaccharidosis IX | Autosome formed |
| 25 | IDS | X | Mucopolysaccharidosis II (Hunter syndrome) | X chromosome |
| | | | | formed |
| 26 | IDUA | 4 | Mucopolysaccharidosis I | Autosome formed |
| | | | (Hyrler, Scheie, and Hurler/Scheie syndromes) | |
| 27 | LAMP2 | X | Danon disease | X chromosome |
| | | | | formed |
| 28 | LIPA | 10 | Wolman disease | Autosome formed |
| | | | Cholesteryl ester storage disease | |
| 29 | MAN2B1 | 19 | α-Mannosidosis | Autosome formed |
| 30 | MANBA | 4 | β-Mannosidosis | Autosome formed |
| 31 | MCOLN1 | 19 | Mucolipidosis IV | Autosome formed |
| 32 | NAGA | 22 | Schindler disease types I and II | Autosome formed |
| | | | (Kanzaki disease) | |
| 33 | NAGLU | 17 | Mucopolysaccharidosis IIIB | Autosome formed |
| | | | (Sanfilippo syndrome B) | |
| 34 | NEU1 | 6 | Sialidosis | Autosome formed |
| 35 | NPC1 | 18 | Niemann-Pick type C disease | Autosome formed |
| 36 | NPC2 | 14 | Niemann-Pick type C disease | Autosome formed |
| 37 | PPT1 | 1 | Neuronal ceroid lipofuscinosis 1 (infantile NCL) | Autosome formed |
| 38 | PSAP | 10 | Gaucher disease | Autosome formed |
| | | | Metachromatic leukodystrophy | |
| 39 | SGSH | 17 | Mucopolysaccharidosis IIIA | Autosome formed |
| | | | (Sanfilippo syndrome A) | |
| 40 | SMPD1 | 11 | Niemann-Pick disease type A and B | Autosome formed |
| 41 | SUMF1 | 3 | Multiple sulfatase deficiency | Autosome formed |
| 42 | TPP1 | 11 | Neuronal ceroid lipofuscinosis 2(Classic | Autosome formed |
| | | | late-infantile NCL) | |

The information about the above genetic patterns is available at Online Mendelian Inheritance in Man database.

Based on the GRCh37/hg19 genomic coordinates, 7,187 germline single nucleotide variants (SNVs) and small insertions and deletions (indels) were identified in protein-coding regions, essential splice junctions, and 5′ and 3′ untranslated regions (UTRs) in the aggregate variant call set of the Pan-Cancer and 1000 Genomes cohorts. Of those, 4,019 (55.9%) were singletons (variants found in only one individual), and 3′ UTR variants accounted for the largest proportion (37.7%).

PPVs were selected based on three different measures to determine their pathogenicity:

(1) predicted mutational effects on the sequence and expression of transcripts and proteins;

(2) clinical and experimental evidence obtained from the curated variant databases such as ClinVar, Human Gene Mutation Database (HGMD) and locus-specific mutation databases (LSMDs) and the medical literature; and

(3) in silico prediction of mutational effects on protein function.

Assuming that variants with a population allele frequency (AF) of 0.5% are extremely unlikely to cause LSDs, variants with an average AF between the Pan-Cancer and 1000 Genomes cohorts higher than this threshold were excluded during the PPV selection process. Using an automated algorithm-based approach, a total of 432 PPVs were selected in 41 genes. No PPV was identified in LAMP2. The selected PPVs were grouped into three tiers with partial overlaps, each tier corresponding to each of the three selection criteria (d of FIG. 1).

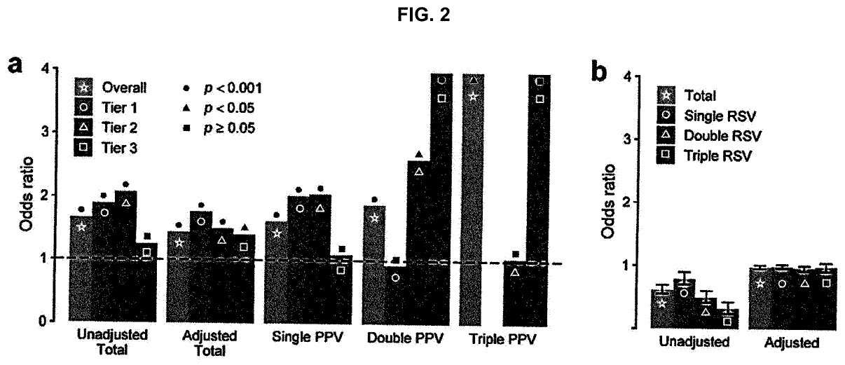

Overall, PPV prevalence was 20.7% in the Pan-Cancer cohort, which was significantly higher than the 13.5% PPV prevalence of the 1000 Genomes cohort (odds ratio, 1.67; 95% confidence interval, 1.44-1.94; P=8.7×10⁻¹²). This association remained significant after adjustment for population structure. The odds ratio for cancer risk was higher in individuals with a greater number of PPVs, and this tendency was broadly consistent when the analysis was restricted to individual tiers (a of FIG. 2). As shown in a of FIG. 2, the odds ratios for double and triple carriers of tier 3 PPVs and triple carriers of total PPVs were 7.54, infinite and 7.4, respectively.

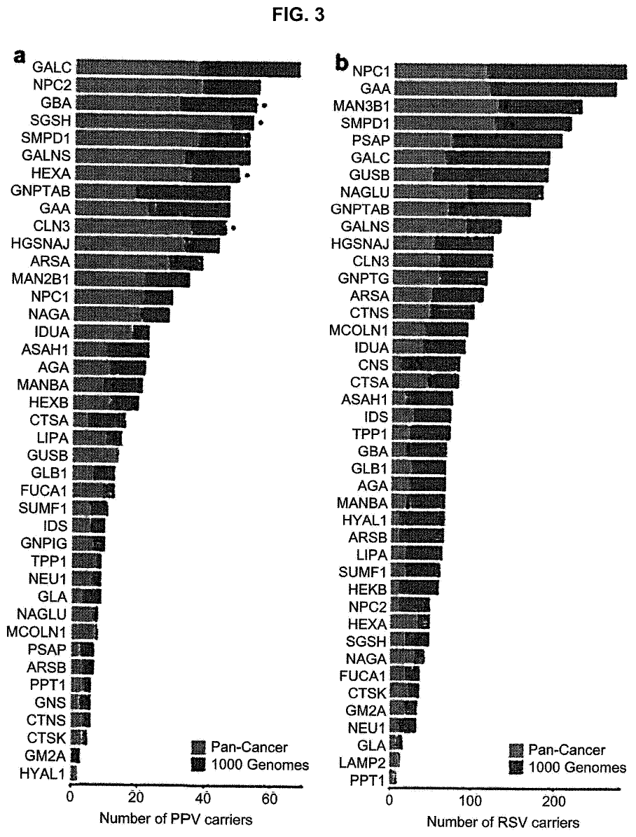

For comparison, the prevalence of rare synonymous variants (RSVs) with an average AF between the Pan-Cancer and 1000 Genomes cohorts of <0.5% was examined. No difference was found between the two cohorts after adjustment for population structure, indicating that the enrichment of PPVs in the Pan-Cancer cohort was not likely due to batch effects (b of FIG. 2). The gene-specific prevalence of PPVs and RSVs in the Pan-Cancer and 1000 Genomes cohorts is shown in FIG. 3.

The results demonstrated that PPVs were relatively more abundant in the Pan-Cancer cohort versus the 1000 Genomes cohort with respect to the abundance of RSVs, for 33 of 42 genes (78.6%; exact binomial test P<0.001).

3. Association of PPVs with Specific Cancer Types

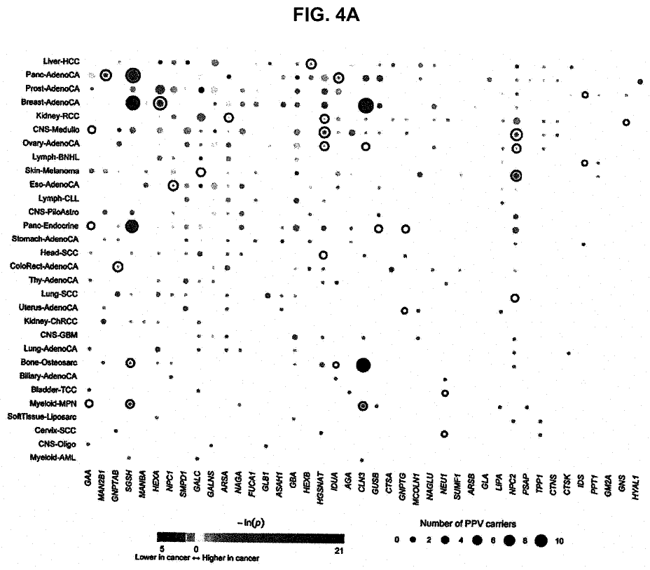

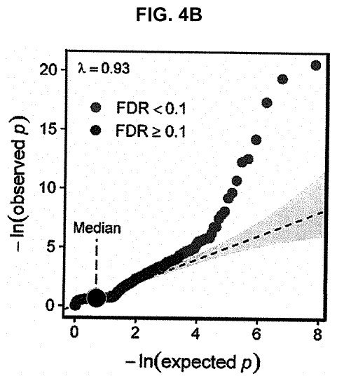

Among the 30 major histological types of cancer (>15 individuals per cancer type), the PPV prevalence ranged from 8.8% to 48.6%, with significantly higher values in seven histological types of cancer than in the 1000 Genomes cohort. The results of tier-based analyses were broadly consistent. In contrast, RSV prevalence showed much less variation across cohorts and was higher in the 1000 Genomes cohort than in any cancer cohort, reflecting the more heterogeneous nature of ancestry and the resulting higher genetic polymorphism in the 1000 Genomes cohort. Analysis using the optimal sequence kernel association test (SKAT-O) method, adjusted for population structure (Methods), unveiled 37 significantly associated cancer-gene pairs and four genes (GBA, SGSH, HEXA and CLN3) with a pan-cancer association (FIG. 4A).

The area of each dot is proportional to the number of PPV carriers for the corresponding cohort-gene pair. Significantly associated cohort-gene pairs at the 0.1 FDR threshold are encircled by bold rings. The cohorts are shown in descending order according to the number of patients they include, and the genes are shown in descending order according to the number of unique PPVs they contain. 19 cancer types were significantly enriched for PPVs in at least one LSD gene, and PPVs in 18 genes were associated with at least one cancer type. A group-based inflation factor (A) is displayed at the top left-hand corner, and gray shading indicates the 95% confidence interval. Each dot in this plot corresponds to each dot shown in FIG. 4A.

### 4. PPV Prevalence in Pan-Cancer and ExAC Cohorts

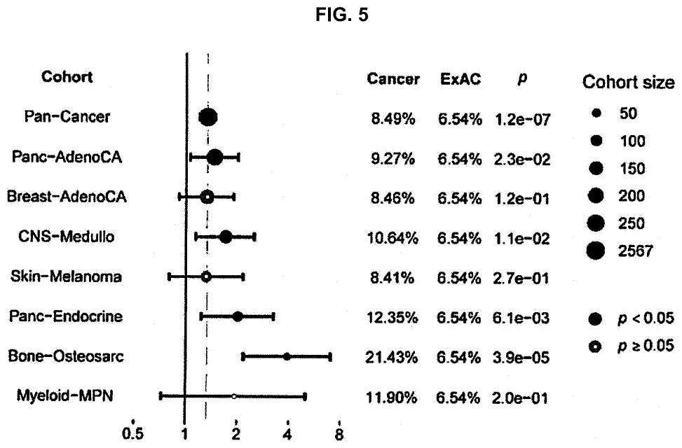

The findings of the SKAT-O analysis were validated using the ExAC cohort as an independent control. For this purpose, focused was placed on (1) eight cancer cohorts that showed significantly higher PPV prevalence than the 1000 Genomes cohort; and (2) ten PPV groups that were significantly enriched in the Pan-Cancer cohort or three or more histological cancer subgroups compared to the 1000 Genomes cohort. As shown in FIG. 5, PPV prevalence was higher in all tested cancer cohorts than in the ExAC cohort, and the association was significant for the Pan-Cancer, pancreatic adenocarcinoma, medulloblastoma, pancreatic neuroendocrine carcinoma, and osteosarcoma cohorts. In addition, all tested PPV groups except GBA were more prevalent in the Pan-Cancer cohort than in the ExAC cohort, and six were significantly enriched in cancer patients.

### 5. Variant-Specific Enrichment of PPVs in Cancer Patients

Among the 432 PPVs identified in the Pan-Cancer and 1000 Genomes cohorts, a splicing variant in NPC2, rs140130028 (ENST00000434013:c.441+1G>A), was most strongly associated with various histological types of cancer including medulloblastoma, ovarian adenocarcinoma, cutaneous melanoma, and lung squamous cell carcinoma. Inactivating mutations of the NPC2 gene cause Niemann-Pick type C disease, which typically presents as progressive neurological abnormalities. The relationship between the Niemann-Pick type C disease and medulloblastoma was implied by a structural homology of NPC1 with Patched transmembrane protein, a tumor suppressor that is regulated by Hedgehog signaling and involved in the development of medulloblastoma when inactivated by loss-of-function mutations.

Vismodegib, a downstream Hedgehog signaling inhibitor, has shown promising antitumor activity in animal models, leading to evaluation of this agent in clinical trials for the treatment of medulloblastoma. Nonetheless, no study to date has provided direct evidence linking medulloblastoma to mutations causing Niemann-Pick type C disease. Results of our study, therefore, provide the first genetic evidence of the tumorigenic potential of inactivating NPC2 mutations.

In addition, rs145834006, a 3′ UTR variant in IDS that was significantly associated with downregulated gene transcription, showed strong association with non-Hodgkin B-cell lymphoma. This finding supports the significant SKAT-O association between IDS PPVs and non-Hodgkin B-cell lymphoma. The relatively high IDS expression in lymphoid tissue implies an essential role of the protein encoded by this gene in lymphoid organ function.

### 6. Age at Diagnosis of Cancer According to PPV Carrier Status

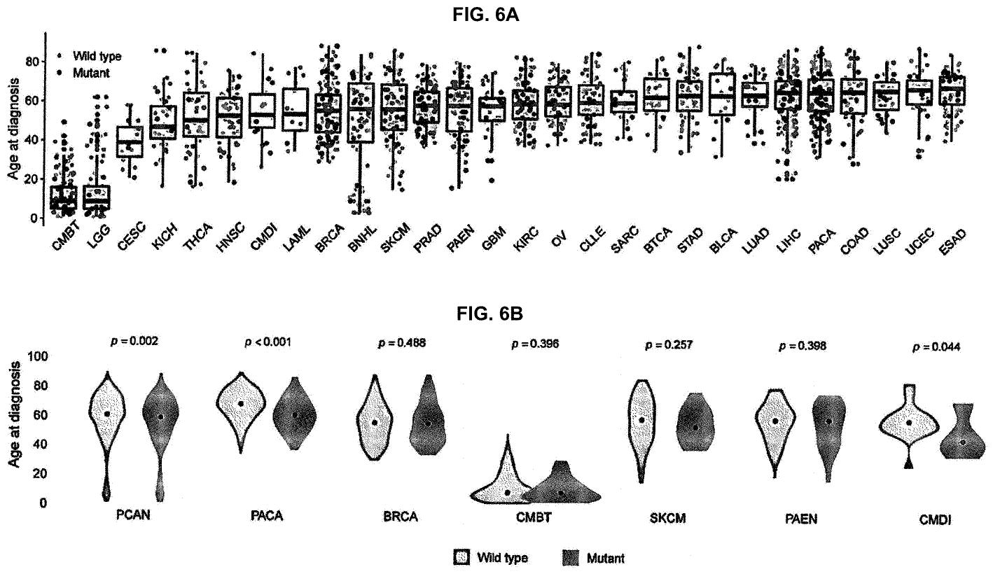

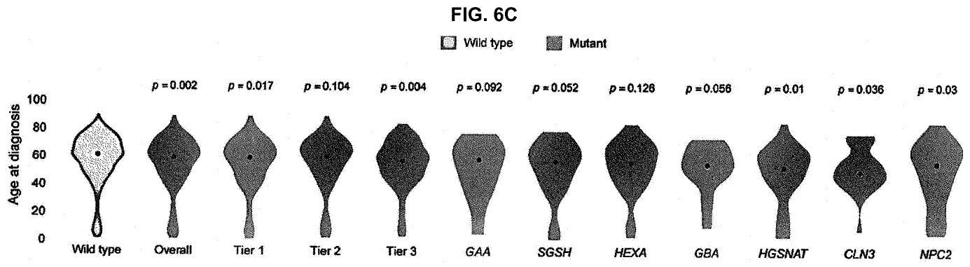

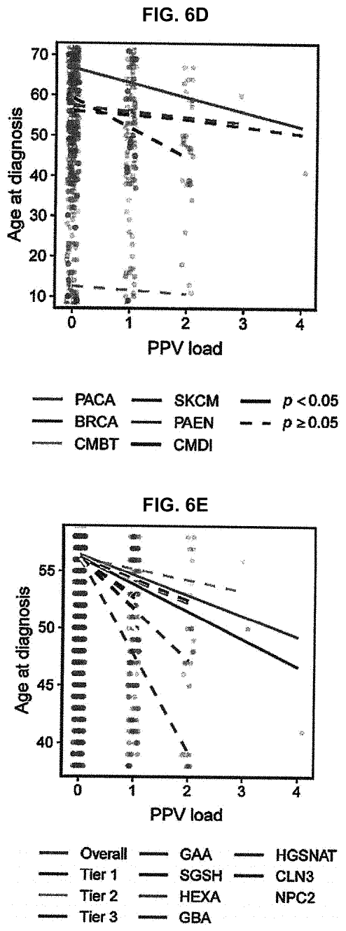

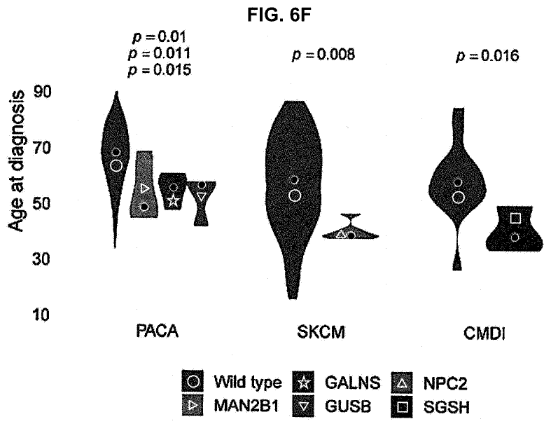

The age at diagnosis of cancer across 28 major clinical cancer cohorts (corresponding to 30 major histological types that included 15 or more patients; information on age at diagnosis was not available for patients with osteosarcoma; patients with pilocytic astrocytoma and oligodendroglioma were combined into a single clinical cohort) is shown in FIG. 6A. In FIG. 6A, patients are represented by red (PPV carrier) or gray (non-carrier) dots. Boxes encompass the 25th through 75th percentiles, the horizontal bar represents the median, and the upper and lower whiskers extend from the upper and lower hinges to the largest and smallest values no further than 1.5× interquartile range from the hinges, respectively.

To examine whether cancer occurred earlier in PPV carriers than in wild-type individuals, the age at diagnosis of cancer was compared according to PPV carrier status in the Pan-Cancer cohort and in six clinical cancer subgroups that showed significant SKAT-O association with PPVs (FIG. 6B). Referring to FIG. 6B, the median age at diagnosis of cancer was numerically lower in PPV carriers in all the evaluated cohorts, and the difference was significant in PCAN, PACA and CMDI.

Next, the age at diagnosis of cancer was compared between carriers and non-carriers of PPVs that belonged to each PPV group that was significantly enriched in the Pan-Cancer cohort or three or more cancer types compared to the 1000 Genomes cohort. The same criteria were used for the validation of SKAT-O results with the ExAC cohort as an independent control (FIG. 6C). As shown in FIG. 6C, the carriers of PPVs that belonged to tier 1, tier 3, HGSNAT, CLN3 and NPC2 showed significantly earlier onset of cancer compared to wild-type (PPV non-carrier) individuals.

Moreover, the PPV load (number of PPVs per individual) showed a consistent negative linear correlation with age at diagnosis of cancer across all histological types and PPV groups evaluated, and the correlation was significant in the Pan-Cancer and pancreatic adenocarcinoma cohorts (FIG. 6D and FIG. 6E). Exploratory analysis across all cancer types and genes revealed earlier cancer onset in PPV carriers for five additional cancer-gene pairs, three of which (pancreatic adenocarcinoma-MAN2B1, cutaneous melanoma-NPC2 and chronic myeloid disorder-SGSH) were in accordance with the SKAT-O results (FIG. 6F). In FIG. 6F, the vertically aligned P-values from top to bottom for PACA correspond to the three genes displayed from left to right, respectively.

### 7. Differential Somatic Mutation and Gene Expression Pattern Patterns of Pancreatic Adenocarcinoma in PPV Carriers

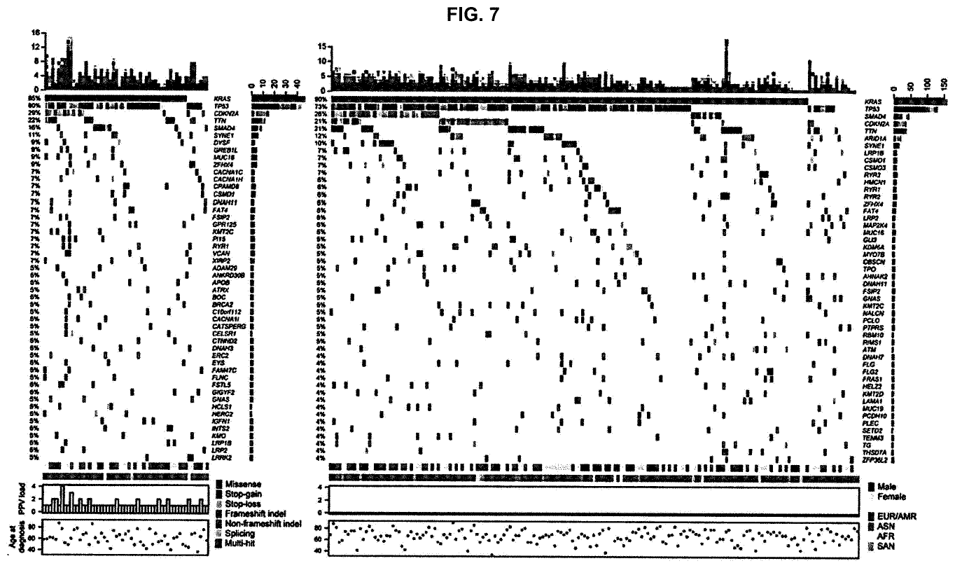

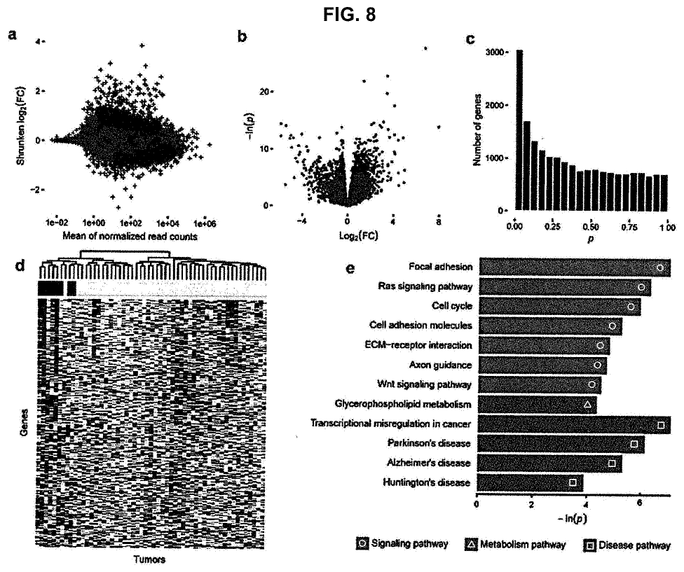

It was investigated whether the differentiating patterns of somatic mutations and gene expression underlie the oncogenic processes triggered by PPVs in pancreatic adenocarcinoma, for which both the SKAT-O analysis and comparison of age at diagnosis of cancer according to PPV carrier status produced consistent results (FIG. 4A, FIG. 6B, FIG. 6D and FIG. 6F). In addition, the somatic mutational landscape was compared between tumors from PPV carriers (n=55) and non-carriers (n=177). The 50 most frequently mutated genes in each group are shown in FIG. 7.

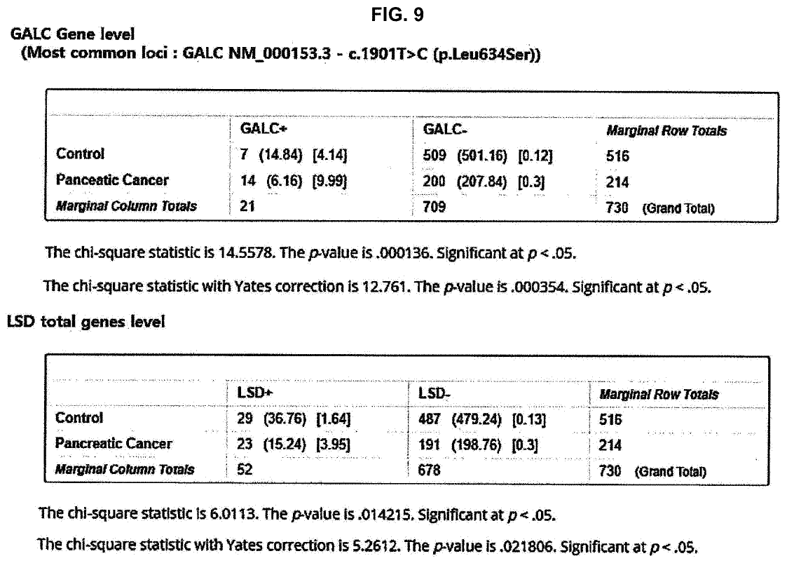

Referring to FIG. 7, KRAS, TP53, CDKN2A, TTN and SMAD4 showed high mutation frequency. The results for KRAS, TP53, CDKN2A and TTN among them were in agreement with the previous genome sequencing studies of pancreatic adenocarcinoma. Non-silent mutation burden was similar between groups (mean 57.1 versus 56.3 mutations per tumor for PPV-associated versus PPV-unrelated cases, respectively; P=0.9). Mutational signature also did not differ according to the PPV carrier status (P≥0.05 for all signatures; Supplementary FIG. 9).

Differentially expressed gene (DEG) analysis of pancreatic adenocarcinoma samples using available RNA-Seq data revealed 287 gene upregulations and 221 downregulations in tumors from PPV carriers compared to those from wild-type individuals (a to d of FIG. 8). In a of FIG. 8 and b of FIG. 8, genes with FDR<0.1 are shown as red dots. In c of FIG. 8, the histogram of P-values shows a peak frequency below 0.05, demonstrating the existence of up- or downregulated genes.

And, in d of FIG. 8, the relative expression of genes significantly up- or downregulated at the 0.1 FDR threshold in tumors from PPV carriers versus non-carriers is labeled with red and gray bars, respectively. The samples were ranked according to the FPKM-UQ-normalized read counts for each gene and the rank numbers were used for color mapping in order to standardize the visual contrast across genes. The samples were ordered as columns by hierarchical clustering based on the Euclidean distance and complete linkage. The genes were ordered as rows in the same manner (dendrogram not shown). High and low relative expression was indicated by progressively more saturated red and blue colors, respectively.

Pathway-based analysis with the generally applicable gene set enrichment (GAGE) method identified 63 pathways significantly altered by PPV carrier status (e of FIG. 8). Remarkably, these pathways included at least six among 13 core signaling pathways that have been shown to be recurrently perturbed in pancreatic cancer (Ras signaling, Wnt signaling, axon guidance, cell cycle regulation, focal adhesion, cell adhesion, and ECM-receptor interaction pathways). In addition, the data suggested that deleterious mutations in LSD genes can provoke perturbations in neurodegenerative disease pathways involved in the development of Parkinson disease, Alzheimer disease, and Huntington disease, all of which have been reported to occur frequently in LSD patients. The glycerophospholipid metabolism pathway was also identified, indicating that altered gene expression and nonsense-mediated decay might have contributed to lysosomal dysfunction in PPV carriers.

### 8. Two-Hit Analysis of Lysosomal Storage Disease Genes in Cancer Cells

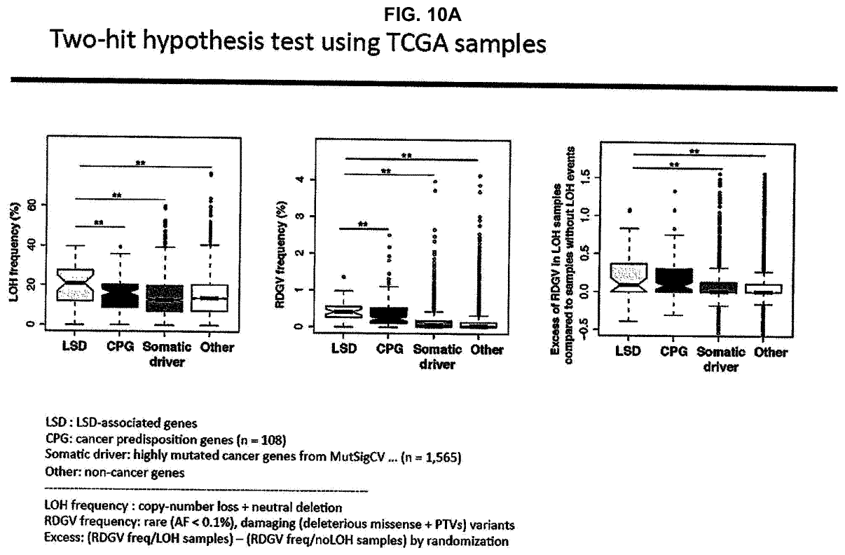

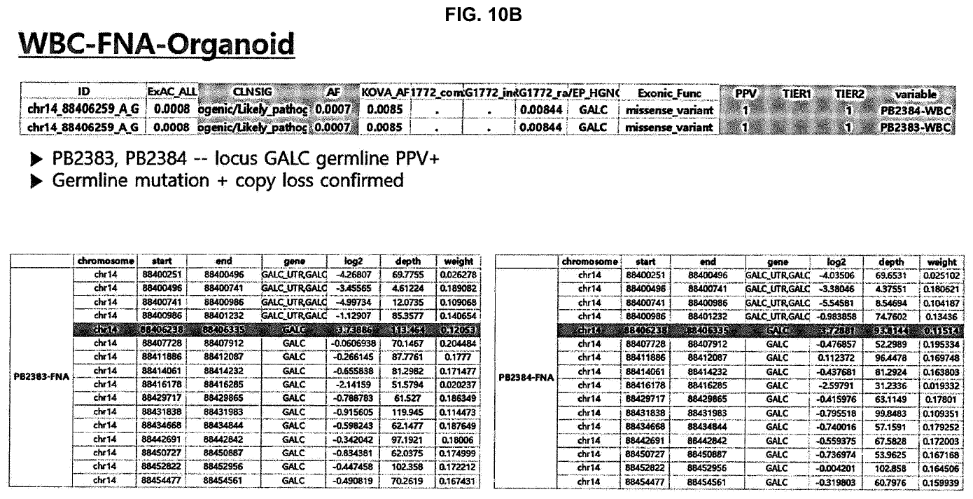

The “two-hit hypothesis” is the hypothesis that cancer occurs as both alleles lose their function due to inactivation. If a second hit occurs in the heterozygote carrier of a specific gene for some reason, the cell may die or develop into cancer on the contrary. In order to confirm this, the inventors of the present disclosure have compared LOH with known cancer predisposition genes using Alfred's method and have obtained a statistically significant result (FIG. 10A). It has been found that many of the carriers of genetic disease-related gene variants occurring with cancer-specifically high frequencies had CN deletion/loss. In addition, somatic variants were found in the same gene of some tumor tissues. The “two-hit” analysis of sex chromosomes required additional comparison according to the gender ratio in each cohort. For example, because a single genetic variation or CNV in the X chromosome can be fatal for men, the gender information of samples is important. For this reason, sex chromosomes were excluded from the analysis.

### 9. Whole Exome Sequencing Data Analysis Results for Korean Pancreatic Cancer Patients

### 9-1. Relationship Between PPVs and Pancreatic Cancer in Germ Cells

The frequency of LSD-related PPVs in germ cells was investigated using the WES (whole exome sequencing) germline data. The result is given in Tables 3 and 4 and visualized in FIG. 9.

TABLE 3

| PPV | Carrier | Non-carrier | Total | Freq |
|---|---|---|---|---|
| PANCREAS WES (214) | 23 | 191 | 214 | 0.107476636 |
| NC (516) | 29 | 487 | 516 | 0.05620155 |

TABLE 4

| | ExAC_ | | | KRG1772_ | VEP_ | Exonic_ | |
|---|---|---|---|---|---|---|---|
| ID | ALL | CLNSIG | AF | rare | HGNC | Func | Sample |
| chr22_51064362_ | . | Likely_pathogenic | . | . | ARSA | splice_ | PB2311 |
| AC_A | | | | | | donor_ | |
| | | | | | | variant | |
| chr20_44523378_ | . | . | 0 | . | CTSA | splice_ | PB1898 |
| TAGGTAGGTG | | | | | | donor_ | |
| CTGCTGGGTG | | | | | | variant | |
| CCCCTGGAGC | | | | | | | |
| CAACCCCAGC | | | | | | | |
| CCCATCTGGA | | | | | | | |
| GGCTCCACAC | | | | | | | |
| CCATTCCCCCA | | | | | | | |
| CCTCACATTGC_ | | | | | | | |
| T | | | | | | | |
| (SEQ ID NO: 1) | | | | | | | |
| chr20_44523537_ | . | . | 0 | . | CTSA | splice_ | PB1898 |
| TCAGGTGTGC | | | | | | donor_ | |
| AGGGCGTGG | | | | | | variant | |
| GCTTCCTCCTG | | | | | | | |
| GTGAGGTGGG | | | | | | | |
| GGCAGGGGGA | | | | | | | |
| GGGGCAGGGA | | | | | | | |
| AGCAGAGGCC | | | | | | | |
| CTGACCCACT | | | | | | | |
| GTCTGTGCCTT | | | | | | | |
| C_T | | | | | | | |
| (SEQ ID NO: 2) | | | | | | | |
| chr17_78078931_ | 3.01E-05 | Pathogenic/Likely_ | 2.96E-05 | . | GAA | synonymous_ | PB2423 |
| G_A | | pathogenic | | | | variant | |
| chr17_78079575_ | . | . | 0 | . | GAA | stop_ | PB1952 |
| G_T | | | | | | gained | |
| chr14_88401093_ | 0.0002 | Likely_pathogenic | 0.0002 | 0.00174723 | GALC | missense_ | PB1262 |
| C_T | | | | | | variant | |
| chr14_88406259_ | 0.0008 | Pathogenic/Likely_ | 0.0007 | 0.00844496 | GALC | missense_ | PB1486 |
| A_G | | pathogenic | | | | variant | |
| chr14_88406259_ | 0.0008 | Pathogenic/Likely_ | 0.0007 | 0.00844496 | GALC | missense_ | PB1926 |
| A_G | | pathogenic | | | | variant | |
| chr14_88406259_ | 0.0008 | Pathogenic/Likely_ | 0.0007 | 0.00844496 | GALC | missense_ | PB2024 |
| A_G | | pathogenic | | | | variant | |
| chr14_88406259_ | 0.0008 | Pathogenic/Likely_ | 0.0007 | 0.00844496 | GALC | missense_ | PB2384- |
| A_G | | pathogenic | | | | variant | WBC |
| chr14_88406259_ | 0.0007 | Pathogenic/Likely_ | 0.0008 | 0.00844496 | GALC | missense_ | PB2383- |
| A_G | | pathogenic | | | | variant | WBC |
| chr14_88406259_ | 0.0008 | Pathogenic/Likely_ | 0.0007 | 0.00844496 | GALC | missense_ | PB402- |
| A_G | | pathogenic | | | | variant | WBC |
| chr14_88406259_ | 0.0008 | Pathogenic/Likely_ | 0.0007 | 0.00844496 | GALC | missense_ | PB576- |
| A_G | | pathogenic | | | | variant | WBC |
| chr14_88406259_ | 0.0008 | Pathogenic/Likely_ | 0.0007 | 0.00844496 | GALC | missense_ | PB1930 |
| A_G | | pathogenic | | | | variant | |
| chr14_88406259_ | 0.0008 | Pathogenic/Likely_ | 0.0007 | 0.00844496 | GALC | missense_ | PB2200 |
| A_G | | pathogenic | | | | variant | |
| chr14_88406259_ | 0.0008 | Pathogenic/Likely_ | 0.0007 | 0.00844496 | GALC | missense_ | PB2222 |
| A_G | | pathogenic | | | | variant | |
| chr14_88406259_ | 0.0008 | Pathogenic/Likely_ | 0.0007 | 0.00844496 | GALC | missense_ | PB1205 |
| A_G | | pathogenic | | | | variant | |
| chr14_88406259_ | 0.0008 | Pathogenic/Likely_ | 0.0007 | 0.00844496 | GALC | missense_ | PB1638 |
| A_G | | pathogenic | | | | variant | |
| chr14_88406259_ | 0.0008 | Pathogenic/Likely_ | 0.0007 | 0.00844496 | GALC | missense_ | PB1636 |
| A_G | | pathogenic | | | | variant | |
| chr14_88406259_ | 0.0008 | Pathogenic/Likely_ | 0.0007 | 0.00844496 | GALC | missense_ | PB1028 |
| A_G | | pathogenic | | | | variant | |
| chr5_74014629_ | 0.0006 | Pathogenic/Likely_ | 0.0006 | 0.00465929 | HEXB | missense_ | PB1929 |
| C_T | | pathogenic | | | | variant | |
| chr5_74014629_ | 0.0006 | Pathogenic/Likely_ | 0.0006 | 0.00465929 | HEXB | missense_ | PB921 |
| C_T | | pathogenic | | | | variant | |
| chr5_74014629 | 0.0006 | Pathogenic/Likely_ | 0.0006 | 0.00465929 | HEXB | missense_ | PB615 - |
| C_T | | pathogenic | | | | variant | |
| chr5_74016342_ | . | . | 0 | . | HEXB | splice_ | PB1898 |
| TGGTATGGGGA | | | | | | acceptor_ | |
| TTTACCTGATA | | | | | | variant; | |
| ACATTTAAGAA | | | | | | splice_ | |
| TTAAGGTGCCT | | | | | | donor_ | |
| TAGCTTTCCTT | | | | | | variant | |
| CTCTGTCTAAA | | | | | | | |
| CACAAAAGTG | | | | | | | |
| CTAAACATAAA | | | | | | | |
| TTTAAACTGCT | | | | | | | |
| TGCGGGGGGA | | | | | | | |
| TGTGTGATTTA | | | | | | | |
| AATTTTA_T | | | | | | | |
| (SEQ ID NO: 3) | | | | | | | |
| chr4_981624_C | . | . | . | . | IDUA | frames | PB1205 |
| CAGTACGTCCT_ | | | | | | hift) | |
| (SEQ ID NO: 4) | | | | | | variant | |
| chr19_12760828_ | . | . | 0 | . | MAN2B1 | splice_ | PB1926 |
| GCTGTACCCA | | | | | | acceptor_ | |
| ATGGGATGGC | | | | | | variant; | |
| AAGGTTGTGA | | | | | | splice_ | |
| GCCTTGGATAA | | | | | | donor_ | |
| ACCCCTCTGC | | | | | | variant | |
| CCTTGCTTCCA | | | | | | | |
| CACCCCTCTC | | | | | | | |
| CCAGCCTGTG | | | | | | | |
| CCACTCAC_G | | | | | | | |
| (SEQ ID NO: 5) | | | | | | | |
| chr18_21141366_ | . | . | 0 | . | NPC1 | frames | PB1097 |
| CCTTATTGA_ | | | | | | hift_variant | |
| C | | | | | | | |
| (SEQ ID NO: 6) | | | | | | | |
| chr10_73577233_ | . | . | 0 | . | PSAP | frames | PB1898 |
| C_CATTGCAC | | | | | | hift_variant | |
| TGGGCTGCTG | | | | | | | |
| TCTCTGTGTTC | | | | | | | |
| TGGCACCAGT | | | | | | | |
| AGCTTGGG | | | | | | | |
| (SEQ ID NO: 7) | | | | | | | |
| chr10_73579379_ | . | . | 0 | . | PSAP | splice_ | PB1898 |
| ACTACATAAG | | | | | | acceptor_ | |
| AGGGCAGCGG | | | | | | variant; | |
| GCTCAACGCT | | | | | | splice_ | |
| GGCAGGGCCC | | | | | | donor_ | |
| TCCCAGACCC | | | | | | variant | |
| AAGAGGGGCA | | | | | | | |
| CCATCCTCTCC | | | | | | | |
| CGCACCACAC | | | | | | | |
| CCAGCGCTCA | | | | | | | |
| C_A | | | | | | | |
| (SEQ ID NO: 8) | | | | | | | |
| chr10_73579379_ | . | . | 0 | . | PSAP | splice_ | PB1926 |
| ACTACATAAG | | | | | | acceptor_ | |
| AGGGCAGCGG | | | | | | variant; | |
| GCTCAACGCT | | | | | | splice_ | |
| GGCAGGGCCC | | | | | | donor_ | |
| TCCCAGACCC | | | | | | variant | |
| AAGAGGGGCA | | | | | | | |
| CCATCCTCTCC | | | | | | | |
| CGCACCACAC | | | | | | | |
| CCAGCGCTCA | | | | | | | |
| C_A | | | | | | | |
| (SEQ ID NO: 8) | | | | | | | |

As shown in FIG. 9, the frequency of PPVs in germ cells was increased in pancreatic cancer, and the odds ratio of GALC gene mutation with pancreatic cancer was 5.09.

### 9-2. Frequency of PPVs in GALC Gene in Pancreatic Cancer Patients

TABLE 5
GALC Carrier

| Cancer Type | PPV count | Total | Frequency |
|---|---|---|---|
| PANCREAS. WES (214) | 15 | 214 | 0.065420561 |
| No history of NC carcinoma (516) | 7 | 516 | 0.013565891 |

GALC “chr14_88406259_A_G” Tier_2 carrier

| Cancer Type | PPV count | Total | Frequency |
|---|---|---|---|
| PANCREAS. WES (214) | 14 | 214 | 0.065420561 |
| No history of NC carcinoma (516) | 7 | 516 | 0.013565891 |

### 10. Two-Hit and Expression Level Data Analysis Results for Korean Pancreatic Cancer Patient Organoids

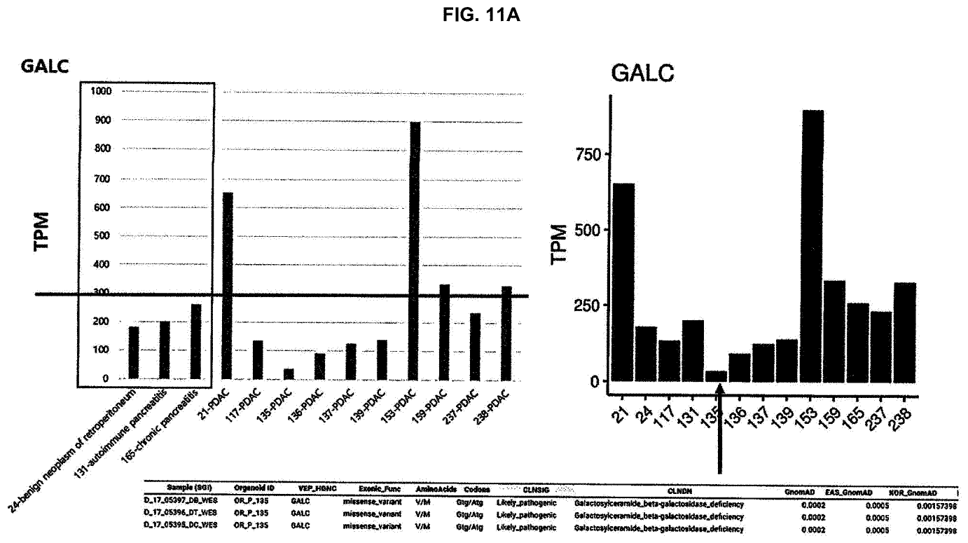

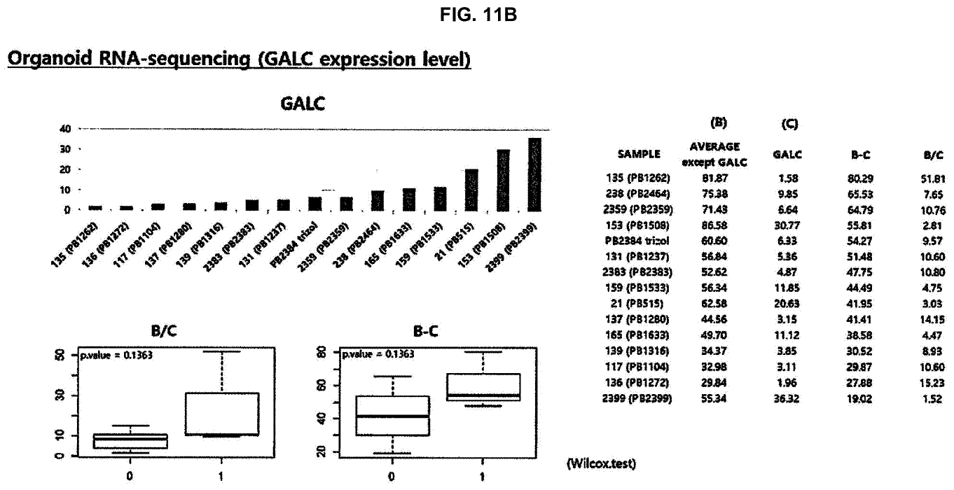

Gene expression analysis and two-hit analysis were conducted on the organoid sequencing data of Korean pancreatic cancer patients. Copy number loss was confirmed in the same regions where genetic variations occurred in the GALC gene PPV carrier organoids (FIG. 10B), and gene expression was significantly decreased as compared to the organoids of non-carriers (FIG. 11A and FIG. 11B). The absolute expression level was compared for each gene using TPM values. In addition, as a result of comparing the expression level of 42 LSD genes and the GALC gene, it was found that the carrier group showed low expression levels.

While the specific exemplary embodiments of the present disclosure have been described above, it will be obvious to those having ordinary knowledge in the art that they are merely preferred exemplary embodiments and the scope of the present disclosure is not limited by them. Accordingly, it is to be understood that the substantial scope of the present disclosure is defined by the appended claims and their equivalents.

By revealing a potential mechanism in which PPVs are related to the occurrence of cancer through analysis of genomic and transcriptomic data of cancer obtained from studies using an Asian cohort with pancreatic adenocarcinoma and an organoid, the inventors of the present disclosure have expanded the scope of understanding about the vulnerability to genetic cancer and established a basis for suggesting that a therapeutic strategy using a technique for reviving lysosomal function may be used for personalized prevention and treatment of cancer.

A sequence listing electronically submitted with the present application on Mar. 30, 2022 as an ASCII text file named 20220330_Q74022DA03_TU_SEQ, created on Mar. 30, 2022 and having a size of 2000 bytes, is incorporated herein by reference in its entirety.

1-11. (canceled)

11: A method for diagnosing a risk of pancreatic cancer, the method comprising
detecting mutation or functional decrease of a gene comprising at least one selected from a group consisting of ARSA (arylsulfatase A), CTSA (cathepsin A), GAA (acid alpha-glucosidase), GALC (galactosylceramidase), HEXB (hexosaminidase subunit beta), IDUA (iduronidase), MAN2B1 (mannosidase alpha class 2B member 1), NPC1 (NPC intracellular cholesterol transporter 1) and PSAP (prosaposin) from a biological sample of a subject; and
determining that there is a higher risk of the pancreatic cancer when the mutation or functional decrease of the one or more gene is detected than when neither mutation decrease nor functional decrease is detected.

12. (canceled)

13: The method of claim 11, wherein the subject is an Asian.

14: The method of claim 11, wherein the biological sample is a blood or a cancerous tissue of the subject.

15: The method of claim 11, wherein the detecting is performed by one or more method selected from a group consisting of measurement of an activity of a protein encoded by the gene, measurement of the expression level of the gene and gene sequencing.

16: The method of claim 11, wherein the determining comprises determining that the risk of pancreatic cancer is 5 times higher when there is mutation or functional decrease of the GALC gene as compared to a normal group with no mutation or functional decrease.

17: The method of claim 11, wherein the determining comprises determining that the risk of pancreatic cancer is 2 times higher when mutation or functional decrease is detected in two or more genes selected from a group consisting of ARSA, CTSA, GAA, GALC, HEXB, IDUA, MAN2B1, NPC1 and PSAP.

18: The method of claim 11, wherein the gene comprises the ARSA (arylsulfatase A).

19: The method of claim 11, wherein the gene comprises the CTSA (cathepsin A).

20: The method of claim 11, wherein the gene comprises the GAA (acid alpha-glucosidase).

21: The method of claim 11, wherein the gene comprises the GALC (galactosylceramidase).

22: The method of claim 11, wherein the gene comprises the HEXB (hexosaminidase subunit beta).

23: The method of claim 11, wherein the gene comprises the IDUA (iduronidase).

24: The method of claim 11, wherein the gene comprises the MAN2B1 (mannosidase alpha class 2B member 1).

25: The method of claim 11, wherein the gene comprises the NPC1 (NPC intracellular cholesterol transporter 1).

26: The method of claim 11, wherein the gene comprises the PSAP (prosaposin).
